(19) United States
(12) Patent Application Publication
(10) Pub. No.: US 2009/0306386 A1
(43) Pub. Date: Dec. 10, 2009

(54) Plant Extract Obtained by an Extraction Method by Means of Solvents of Plant Origin

(76) Inventors: Hervé André Gérard Durand, London (GB); Fabien Durand, Les Martres de Veyre (FR)

Correspondence Address: OLIFF & BERRIDGE, PLC, P.O. BOX 320850, ALEXANDRIA, VA, 22320-4850, US

(21) Appl. No.: 12/084,432

(22) PCT Filed: Nov. 10, 2006
(86) PCT No.: PCT/FR2006/002506

§ 371 Date: Jun. 17, 2009

(30) Foreign Application Priority Data
Nov. 10, 2005 (FR) 0511451

(51) Int. Cl. C07D 311/30; C07D 471/14

(52) U.S. Cl. 546/41; 549/403

(57) ABSTRACT

The invention relates to a plant extract obtained by a single-phase extraction method, by means of a solvent and/or a mixture of solvents of plant origin, containing characterised and chemically defined constituents. Said plant extract is characterised in that it is free of polyphenol, anthocyan and/or tannin, and comprises a fraction of lipophile-type components and/or a fraction of polar-type components. The invention also relates to an extraction method which enables an inventive extract to be obtained, said method being a single-phase extraction method characterised in that it comprises the following steps: a plant material is brought into contact with a solvent and/or a mixture of solvents of plant origin, containing characterised and chemically defined constituents; the plant material is eliminated; and the solvent and/or mixture of solvents is partially or completely eliminated. Said solvent and/or at least one of the constituents of the mixture of solvents is selected from the group of terpenic solvents having a purity which is at least equal to 98% and being depleted of peroxides.

The invention relates to the field of processes for extracting active compounds from starting materials of natural origin which are used in the formulation of pharmaceutical, cosmetic or food compositions and/or organic chemistry and in particular the chemistry of compounds of natural origin.

Plants and natural products are the source of molecules which can be used in the form of extracts in numerous fields: pharmaceutical, cosmetic or food. The extracts can be therapeutic extracts (active principles of plant origin: digitalin, morphine, and the like), extracts exhibiting a specific cosmetic activity (cell regeneration, antiaging activity) or extracts of substances used as additives (dyes: cochineal red, β-carotene, curcumin; antioxidants: rosemary extracts; preservatives: grapefruit seed extract) or for other applications, such as natural insecticides (pyrethrins, rotenone).

Drying was certainly the first means used to retain the activity of plants but this technique does not make it possible either to concentrate or to protect the active compounds from oxidation.

With the passage of time, numerous methods, such as extraction with water, maceration, steam distillation, leaching, decoction and solvent extraction, have been used to obtain plant extracts.

Steam distillation is a conventional technique limited to the extraction of essential oils. This process uses a large amount of energy to produce the steam which entrains the volatile compounds and the water necessary to condense the vapors is discharged in the form of hot effluent, also resulting in energy losses. The essential oils thus obtained are subjected to temperatures in the region of 100° C. and components sensitive to heat or to hydrolysis are often detrimentally affected.

Among “natural” extraction processes, the Chinese pharmacopeia reports the use of specific solvents, such as wine, alcohol, vinegar, lemon juice, milk or cream, but the extracts obtained using these methods are difficult to characterize.

Extraction employing synthetic organic solvents or organic solvents resulting from petrochemical refining processes is the method which is the most important from the industrial viewpoint due to the standardization of the methods, the need for reproducibility and characterization of the extracts obtained.

Among these processes, those employing water, alcohol or aqueous/alcohol mixtures make it possible to obtain essentially hydrophilic (or highly polar) molecules but these solvents also entrain numerous molecules of little interest which can reduce the activity of the extracts (proteins which cause precipitation, tannins, inorganics) or harm their stability (anthocyans).

Other solvents, such as glycerol and glycol derivatives, used in particular in cosmetics, have a very low solvating power and thus the extracts obtained are poor in active molecules.

Mention will also be made, among organic solvents conventionally employed, of glycol ethers, hexane, ether, acetone, methylene chloride, alcohols, aliphatic or aromatic hydrocarbons, chlorinated and fluorinated solvents or synthetic molecules, such as N-methylpyrrolidone or dimethoxymethane, for example.

These solvents, while they make it possible to obtain lipophilic (or highly nonpolar) molecules with satisfactory yields, exhibit numerous disadvantages. They are in particular highly inflammable and dangerous or extremely toxic to man and his environment.

Synthetic solvents, such as perchloroethylene, methylene chloride, chlorinated solvents and certain fractions resulting from oil, such as hexane, have been very widely used for the extraction of flavorings, fragrances, colorants and active principles from plants. These organic solvents, which are often highly toxic, comprising carcinogenic impurities or denaturants, are particularly targeted by international regulations, which impose very low residual solvent contents (of the order of 1 to 5 ppm). These regulations, which limit or ban their use in food or therapeutic applications, involve expensive reprocessing operations in order to remove the residual solvents, such as additional heating, and the active principles may be decomposed. Furthermore, when they are badly chosen or chosen solely on economic grounds, the chemical nature of these solvents may render the active products unstable.

For some years, certain organic solvents which are only very slightly toxic, such as fluorinated solvents, N-methylpyrrolidone or dimethoxymethane, have been used. Unfortunately, these are not very readily available or have an extremely high cost which limits their use in industry.

Furthermore, as regards regulations in terms of biological certification, no biological certification or seal of approval can be bestowed on plant extracts obtained by processes employing synthetic solvents or solvents resulting from the petrochemical industry, while the demand for this market is increasingly important.

This is because, apart from the constraints of the “biological” nature of the plant to be extracted, biological certification allows only the use of extracts obtained by steam distillation (essential oils), by maceration in a biologically certified excipient or by extraction with CO₂ in the supercritical state.

Alcoholic or aqueous/alcoholic extraction makes it possible to obtain hydrophilic compounds but does not make possible the efficient extraction of nonpolar lipophilic compounds and can result in the extraction of undesirable compounds, such as tannins, which are highly soluble in water.

Extraction by maceration in a vegetable oil or an aqueous/glycerol solution limits the process to the extraction of lipophilic active principles, with yields which are often rather mediocre.

Furthermore, it is difficult to separate the extracted molecules from the extraction excipients, which are thus necessarily incorporated in this form in the formulated products and are capable of thus limiting or modifying the effectiveness of the molecules extracted.

Thus, when an extract has to be able to obtain biological certification, the only technique which can be truly used to obtain an extract comprising non-steam-distillable lipophilic molecules is the technology of extraction by a supercritical fluid. This technology requires specific equipment which makes it possible to create the temperature and pressure conditions under which the gas employed, carbon dioxide or nitrogen, is found in the supercritical state. This technology requires major commitments of capital and very high operating costs which restrict it to extracts of very high value (aromatic principles for perfumery, pharmaceutical specialties). In fact, very few cosmetic active principles are obtained by extraction with supercritical CO₂ and many active principles with a lipophilic nature which cannot be steam distilled are unavailable.

Le Gall (patent FR 77 36976) describes a process for the extraction of organic compounds having therapeutic activity present in plants and products thus obtained, characterized in that a preground plant is treated with natural essences taken from the group consisting of essence of citral, essence of bergamot, essence of mint and essence of rosemary.

The essence used for the extraction of the plants is distilled beforehand by steam distillation under reduced pressure, so as to use only the low-boiling-point fraction, that is to say the fraction distilling at most at 95° C. under 20 mm of mercury, but this fraction is nevertheless composed of a mixture of uncharacterized chemical entities, resulting in the impossibility of characterizing the extract and of obtaining a purified extract by removal of the residual solvent. Moreover, this removal is neither described nor suggested.

Mengal (patent FR 99 13241) describes a process for extraction by a supercritical fluid using a cosolvent, said cosolvent being an excipient which participates directly in the pharmaceutical, cosmetic or food compositions in which the extracts obtained would be incorporated. Terpene compounds are designated among these cosolvents.

Although this process makes it possible to limit the cost price and the complexity of the process for extraction with a supercritical fluid for some active principles, this technique does not make it possible to obtain a pure, characterized and universally employable extract as active principles are not isolated from the excipient.

Grinda (patent FR 79 03590) describes a process for the extraction of insecticidal substances from plants. The process described consists in extracting the active principles from the plants using organic liquids with a low vapor pressure, the boiling points of which are greater than 100° C. and the toxicity of which is low, said liquids not being intended to be removed; terpenes are mentioned, although they dissolve the plant insecticides less well than esters of aromatic acids.

The lipophilicity of the compounds extracted is not discussed and, in the implementation of the process, the high-boiling-point liquid is mixed with a light solvent of known type in order to carry out the extraction, said light solvent being either a synthetic solvent or a solvent resulting from the petrochemical industry.

Pisacane (patent application PCT WO 01/07135 A2) describes a process for the extraction of flowers and plants of the chrysanthemum family and of the Helianthus family in order to produce insecticidal substances. Said insecticidal substances are pyrethrins, compounds which are hydrophobic and insoluble in water. The process described employs solvents extracted from plants, such as terpenes, in a phase of maceration at ambient temperature (20° C.) for about ten minutes with mechanical stirring. After this maceration phase, the solid residue is filtered off and the solvent can optionally be removed or reduced by evaporation or distillation, but can be retained in order to be used as vehicle or carrier for the activity after formulation.

The properties of the compounds extracted, namely their lipophilic or hydrophilic nature and/or their purity, are not discussed.

Nippon Terupen Kagaku (JP 01 290659 A) describes a method for the manufacture of a natural condensed carotene. A corn or peanut oil is hydrolyzed; the hydrolysate is subsequently extracted with a synthetic solvent (hexane or petroleum ether); the latter is then removed and then the carotene is extracted with d-limonene or orange essential oils. Nevertheless, this process exhibits the disadvantage of comprising several stages, the first of which makes use of synthetic solvents, which may be toxic.

Mamidipally Pavan et al (First approach on rice bran oil extraction using limonene) describe the use of limonene as alternative solvent for the extraction of certain oils, in comparison with hexane. Nevertheless, the extractions are performed at high temperatures (approximately 160° C. for d-limonene), which can then cause the active ingredients present in the extracts obtained to be evaporated and/or detrimentally affected.

Rosenberg M. et al. (Carotenoid base food colorant extracted from orange peel by d-limonene-extraction-process and use) describe a method for the extraction of carotenoids comprising two stages (each of 2 minutes) at a temperature of 20° C., in which butylated hydroxytoluene (BHT), a synthetic substance, was added to the d-limonene used as extraction solvent.

Thus, despite the existence of numerous extraction processes, there exists a need for extraction processes which make it possible to obtain plant extracts at costs compatible with the requirements of the market for active principles and which allow said extracts to meet the requirements for biological certification, while not exhibiting the abovementioned disadvantages.

Crude solvents of plant origin are used to obtain products which are not pure but which exhibit, on the contrary, numerous impurities, including those present in the starting extraction solvent itself, peroxides, unidentified molecules, and the like. Thus, no solvent of the monoterpene hydrocarbon type of natural origin and which is chemically defined has thus ever been used for the manufacture of pure extracts.

The present invention makes it possible to meet these requirements and consists of an alternative process for the extraction and purification of molecules having a lipophilic and/or partially polar nature which employs a solvent and/or a mixture of solvents of plant origin, the constituents of which are characterized and chemically defined. Throughout the description, the general term “solvent” will be employed and it will encompass simultaneously solvents of plant origin (they will be isolated and pure molecules) and simultaneously mixtures of solvents of plant origin, the constituents of which are characterized, that is to say all identified, and chemically defined, that is to say pure.

Extraction is carried out using plant material which can be highly varied. It can relate to leaves, stems, roots, radicles, rhizomes, flowers, flower heads, grains, seeds, kernels, pips, peel, trunk fragments, and the like, of various plant species which can, beforehand, have been dried and/or ground and/or mixed and the like. These can be used alone or as a mixture.

In addition to being simple and inexpensive, the process makes it possible to obtain extracts rich in molecules having a lipophilic nature but, surprisingly and unexpectedly, it also makes possible the concomitant extraction of molecules having a polar nature which participate in the activity of the whole plants.

The present invention relates to a plant extract obtained by a single-phase extraction process, characterized in that it comprises the following stages:
bringing a plant material into contact with a solvent and/or a mixture of solvents of plant origin, the constituent or constituents of which are characterized and chemically defined,
removing the plant material,
and partially or completely removing the solvent and/or mixture of solvents,

said solvent and/or at least one of the constituents of said mixture of solvents being chosen from the group consisting of terpene solvents, having a purity at least equal to 98%, and being poor in peroxides.

The present invention relates to a plant extract obtained by a single-phase extraction process as described above, characterized in that it is devoid of polyphenol, of anthocyan and/or of tannin and in that it comprises a fraction of compounds having a lipophilic nature and/or a fraction of compounds having a polar nature. In one embodiment, the extract comprises at most 0.1% of tannins.

Tannins are water-soluble phenolic compounds having a molecular weight of between 500 and 3000 which exhibit, in addition to the conventional reactions of phenols, the property of precipitating alkaloids, gelatin and other proteins.

These polyphenols (or phenol polymers) are polyesters of gallic and ellagic acids predominantly.

Other hydrophilic compounds are often a nuisance during extraction phases, such as anthocyans, which are proanthocyanidol polymers.

The invention also relates to an extract as defined above, characterized in that the compounds having a lipophilic nature are chosen in particular from nonsaponifiable compounds.

Nonsaponifiable compounds are the nonglyceride constituents of oils. They represent from 0.3 to 2% of oils: these are hydrocarbons, carotenoids, sterols (sitosterol, stigmasterol, campesterol), tocopherols (vitamin E), aliphatic alcohols of high molecular weight or terpene alcohols.

Mention will be made, by way of example, of the following compounds having a lipophilic nature:
β-sitosterol, campesterol, stigmasterol, cholesterol and their derivatives, for example green coffee extracts,
curcumin and curcuminoids, for example turmeric extracts,
carotenoids, for example carrot extracts, lycopene, for example tomato extract,
sesquiterpene lactones and helenanin, for example Arnica extracts,
plant alkaloids, in particular chelidonine, extracted from celandine; sanguinarine, chelerythrine, coptisine and berberine, extracted from bloodroot,
free xanthones, for example extracted from gentian,
parthenolides and flavonoid aglycones, for example extracted from feverfew.

It also relates to an extract as defined above, characterized in that the compounds having a polar nature are chosen from alkaloids, neoxanthins, glycolyzed xanthones, flavonoids or glycosylated flavonoids.

Mention will be made, among alkaloids, of caffeine, theophylline and/or theobromine.

Mention will be made, among glycosylated xanthones, of gentioside from gentian.

Mention will be made, among glycosylated flavonoids, of apigenin glycoside from feverfew.

The invention relates to a plant extract obtained directly using a process as defined above, characterized in that it comprises at most 0.1% of tannins.

It also relates to a green coffee extract, characterized in that it comprises at least 0.8% of nonsaponifiable compounds, at least 2.0% of caffeine and at most 0.1% of tannins.

It also relates to a turmeric extract, characterized in that it comprises at least 8% of curcuminoids and at most 0.1% of tannins.

It also relates to an extract of Arnica montana flowers, characterized in that it comprises at least 5% of sesquiterpene lactones, expressed as helenalin, and at most 0.1% of tannins.

It also relates to a celandine extract, characterized in that it comprises at least 0.75% of total alkaloids, expressed as chelidonine or crystals of chelidonine hydrochloride, and at most 0.1% of tannins.

It also relates to a bloodroot extract, characterized in that it comprises crystals of sanguinarine hydrochloride and at most 0.1% of tannins.

It also relates to a gentian extract, characterized in that it comprises at least 3% of free xanthones with at least 0.5% of gentioside and at most 0.1% of tannins.

It also relates to a feverfew extract, characterized in that it comprises at least 0.83% of parthenolides, flavonoid aglycones, apigenin glucosides and at most 0.1% of tannins.

The present invention also relates to a single-phase extraction process, characterized in that it comprises the following stages:
bringing a plant material into contact with a solvent and/or a mixture of solvents of plant origin, the constituent or constituents of which are characterized and chemically defined,
removing the plant material,
and partially or completely removing the solvent and/or mixture of solvents,

said solvent and/or at least one of the constituents of said mixture of solvents being chosen from the group consisting of terpene solvents, having a purity at least equal to 98%, and being poor in peroxides.

In one embodiment, the stage of bringing the plant material into contact with the solvent and/or mixture of solvents is a maceration of the plant material with the solvent and/or mixture of solvents. It can also be a quenching, a spraying, or the like.

In one embodiment, the stage of removing the plant material is a filtration of the plant material.

The constituent or constituents of the solvent according to the present invention are characterized: the solvent is of plant origin and it is composed of terpenes which are all identified.

Terpene solvents constituent a class of isoprenoid hydrocarbons which are the main constituents of numerous plant essences or essential oils.

The precursor of all terpenes is mevalonic acid. All terpenes can be formally broken down to give isoprene units. The main terpenes are pinene, camphene and limonene, and their isomers, but also orange terpenes, pine terpenes, and their oxygen-comprising compounds, linalool or terpineol, for example, or paramenthane.

In one embodiment, the invention thus relates to a single-phase extraction process with a solvent and/or a mixture of solvents of plant origin, characterized in that the solvent and/or mixture of solvents is an isomer or a mixture of isomers of limonene and/or a pinene, alone or as a mixture.

In one embodiment, the solvent used is a mixture of d-limonene and of α-pinene.

In one embodiment, the solvent used is a mixture of d-limonene and of α-pinene, with at least 50% of d-limonene.

In a specific embodiment, the mixture comprises 70% of d-limonene and 30% of α-pinene.

In one embodiment, ethanol can be used as a mixture with at least one terpene solvent.

In specific embodiments, the solvents used will be binary or ternary mixtures, such as, for example:
d-limonene/95° ethanol: 70%/30%,
α-pinene/95° ethand: 70%/30%,
d-limonene/α-pinene/95° ethanol: 50%/201%/30%.

d-Limonene is a natural compound of the family of the monoterpenes which is present in quantity in the peel of citrus fruits. It is a by-product of the fruit juice industry. In this sense, it is abundantly cheap. This monoterpene is also a hydrocarbon having a very high solvating power.

α-Pinene is also produced on a large scale from essences of pine (turpentine) by the wood and paper industry.

In the present invention, the d-limonene and the α-pinene used refer to isolated and characterized molecules. They do not refer to the commercial names of d-limonene and α-pinene commonly used. This is because, resulting from the fruit juice industry, the wood industry or the deterpenation of essential oils, the by-products, which are often mixtures of different terpenes effectively comprising a large portion of d-limonene or of α-pinene, are often sold under the inappropriate name “d-limonene” and “α-pinene”, as in reality mixtures of varied terpenes are involved which additionally comprise numerous impurities (impurities originating from the transportation, oxidized derivatives, waxes, fats and other nonvolatile residues, peroxides). These solvents are used in the prior art to prepare extracts or products exhibiting relatively low degrees of purity.

In that which follows, the term “d-limonene” and the term “α-pinene” denote solvents chemically defined with reference to the chemical nature of said molecules, which are isolated and characterized to a degree of purity of approximately 100%, at least greater than 98%.

d-Limonene and α-pinene of industrial grade can be readily purchased, for example from the following companies: Capua Sri, Campo Calabro, Italy, or Les Dérivédsésiniqueset Terpéniques, Dax, France, it being possible for these same solvents of biological grade to be purchased, for example from the companies: Montecitrus, Monte Azul Paulista, Brazil, or Sirius, Paris, France, with a purity at least equal to 98%.

The solvents originating from these industrial sources comprise in particular a low portion of impurities composed of oxidation products of d-limonene or of α-pinene, which represents between 0.3 and 0.6% by weight of the products. Depending on the applications envisaged, this fraction, nonvolatile at 40° C. at atmospheric pressure, may or may not be removed, for example by azeotropic steam distillation of the d-limonene or of the α-pinene, which makes it possible to obtain solvents pure to at least 98%, indeed even to at least 99%, indeed even to 99.95%.

The constituent or constituents of the solvent according to the present invention are chemically defined: the terpene solvent used according to the present invention exhibits a very high degree of purity and a very low content of peroxides.

In a specific embodiment, the solvent and/or the constituent or constituents of the mixture of solvents has been subjected to a preliminary purification stage. This purification stage can, for example, be chosen from azeotropic steam distillation or from vacuum evaporation.

The terpene solvent and/or at least one of the constituents of the mixture of solvents exhibits a degree of purity at least equal to 98%. In one embodiment, the degree of purity is greater than or equal to 99%. In a specific example, the degree of purity is 99.95%.

The terpene solvent and/or at least one of the constituents of the mixture of solvents is also very poor in peroxides. This characteristic is fundamental as the formation of peroxides, which occurs during any mixing or heating operation during the stages of the extraction, is initiated by the peroxides already present in the solvent. These same peroxides can detrimentally affect the advantageous compounds. Thus, the more the terpene solvent is rich in peroxides at the start, the greater will be its tendency to decompose (radical runaway effect). Conversely, the poorer the solvent is in peroxides at the start, the less it will be peroxidized during the extraction and the more easily it can be recycled at the end of the process.

In one embodiment, the content of peroxides is less than 15 meq/kg. In another embodiment, the content of peroxides is less than 11.1 meq/kg.

The level of nonvolatile residue is also very low. In one embodiment, this level is less than 0.1%. In another embodiment, this level is less than 0.01%.

The solvents of plant origin, the constituent or constituents of which are characterized and chemically defined, are biodegradable, relatively nonflammable, nontoxic and environmentally friendly. In addition, they are easy to use: dilutable and miscible with water with conventional surfactants.

Although these solvents of plant origin exhibit all the characteristics required in terms of harmlessness, it may be necessary to remove any trace of these from the plant extracts obtained in order to obtain the active principles in the pure form.

These terpene solvents can be removed by various techniques:
by azeotropic steam distillation at 35° C. under a vacuum of 2000 Pa (15 mmHg). Injection of steam into the filtrate makes it possible to remove from 97.26 to 99.99% of the d-limonene and from 98.2 to 99.99% of the α-pinene;
using binary (d-limonene/ethanol) or ternary (d-limonene-/pinene/ethanol) mixtures, the removal of the monoterpene hydrocarbons is facilitated since 99.2% and 99.6% respectively of the solvent mixtures are then removed;
by molecular distillation under very high vacuum between 0.1 and 0.01 Pa (7.5×10⁻⁴ and 7.5×10⁻⁶ mmHg), which makes it possible to remove 99.99% of the d-limonene.

The invention thus also relates to the process according to the invention, characterized in that the stage of removal of the solvent is a stage of azeotropic steam distillation.

The invention also relates to the process according to the invention, characterized in that the stage of removal of the solvent is a vacuum molecular distillation.

These solvents can subsequently be easily recycled for other extractions, in particular due to their low content of peroxides.

The process according to the invention is carried out with plant matter/solvent (plant material/monoterpene hydrocarbon) proportions by weight of 1/4 to 1/10 .

In one embodiment, the extraction temperature is between 40 and 45° C. The duration of maceration can be approximately 3 h 30.

In one alternative form, a stage of irradiation with microwaves is carried out during the maceration. This stage can be carried out at 100 W for 2 minutes 30 seconds. This makes possible a reduction in the extraction time.

The virtually colorless and odorless d-limonene or α-pinene used in the examples below exhibit a purity of greater than 99.95% (analysis by gas chromatography)

### EXAMPLE No. 1

### Simultaneous Extraction of the Nonsaponifiable Compounds and Caffeine from Green Coffee and Removal of the Solvent at Low Temperature by Azeotropic Steam Distillation Under Vacuum

Green coffee usually comprises 14% of lipids, 0.1% of nonsaponifiable compounds and at least 1% of caffeine. 1.5 kg of green coffee beans of biological grade, ground beforehand, are macerated in 15 liters of d-limonene at 40° C. for 3 h 30 with stirring (plant material/solvent ratio: 1/10).

The particles are removed by filtration in order to obtain a solution laden with advantageous compounds, from which the d-limonene is subsequently removed by azeotropic steam distillation. The entire device is placed under vacuum (10-40 mmHg, i.e. 1300-5300 Pa), which makes it possible to considerably lower the temperatures employed and facilitates the evaporation of the solvent: a boiler makes it possible to produce steam at 30° C. which is injected at the surface or into the extract to be desolvated, itself heated to 45° C. The d-limonene is evaporated and then recycled, using a condenser maintained at 0° C., for subsequent extraction operations, while the nonsaponifiable compounds are recovered in the extractor.

18.7 liters of water are thus evaporated in order to remove 15 liters of d-limonene. 188.1 g of extract rich in lipid compounds, in nonsaponifiable compounds and in caffeine are thus obtained. The extraction yield, expressed with respect to the dry weight of plant, is 13.43%.

The same maceration and then desolvation operations can be carried out with different solvents or mixtures of solvents and the extraction yields as follows, expressed with respect to the dry weight of plant, are obtained:

| | |
|---|---|
| Solvent 1: hexane | 11.7% |
| Solvent 2: d-limonene | 13.43% |
| Solvent 3: d-limonene/95° alcohol mixture as a | 13.79% |
| 70/30 (v/v) mixture | |
| Solvent 4: d-limonene of biological grade | 14.07% |
| Solvent 5: α-pinene | 13.65% |
| Solvent 6: α-pinene of biological grade | 14.72% |
| Solvent 7: d-limonene/α-pinene 70/30 (v/v) mixture: | 14.52% |

The extraction yields obtained with monoterpene hydrocarbons are better than those obtained with hexane. The best extraction yields are obtained with biological α-pinene, the d-limonene/α-pinene 70/30 (v/v) mixture, biological d-limonene and the d-limonene/95° alcohol mixture as a 70/30 (v/v) mixture. A synergistic effect of the mixture of solvents of d-limonene/α-pinene 70/30 (v/v) is observed.

The extracts thus obtained comprise less than 0.05% of residual solvent, this limit being unacceptable for the extract obtained using hexane.

The nonsaponifiable compounds and caffeine formed the subject of an analytical study which allows the following extraction yields to be expressed:

| | Nonsaponifiable compounds | Caffeine |
|---|---|---|
| Solvent 1: | 0.815% | nondetectable |
| Solvent 2: | 0.895% | 2.53% |
| Solvent 3: | 0.825% | 3.68% |
| Solvent 4: | 0.912% | 2.47% |
| Solvent 5: | 0.789% | 2.64% |
| Solvent 6: | 0.805% | 2.74% |
| Solvent 7: | 0.952% | 3.98% |

The content of tannins, determined by the method described in the European Pharmacopeia 5.02 notice “Determination of tannins in herbal drugs (2.8.14.)”, is less than 0.1%.

The above method thus makes it possible to selectively extract the nonsaponifiable compounds and the caffeine and to concentrate them.

This exemplary embodiment demonstrates one of the characteristics of the invention, that is to say that monoterpene hydrocarbons make possible the concomitant extraction of compounds having a lipophilic nature (example: nonsaponifiable compounds) and of compounds having a polar nature (example: caffeine) and their concentration, while excluding other components (polyphenols, tannins) nevertheless present in a large amount in the starting material.

The process according to the invention effectively makes possible the selective extraction of lipophilic compounds and compounds of intermediate polarity. The extract thus obtained, rich in nonsaponifiable compounds and in caffeine, can be directly incorporated in dermopharmaceutical formulations, thus reducing the solubilization stages often necessary during the introduction of active principles into topical formulations.

During the maceration stage, it is not necessary to replace the solvent as the d-limonene virtually completely exhausts the green coffee: a single extraction is necessary. It is also possible to extract the nonsaponifiable compounds of green coffee and caffeine using a continuous countercurrentwise extraction device.

### EXAMPLE No. 2

### Simultaneous Extraction of the Nonsaponifiable Compounds and Caffeine from Green Coffee and Removal of the Solvent at Low Temperature Under Vacuum in the Absence of Water

1.5 kg of green coffee of biological grade, ground beforehand, are macerated in 15 liters of d-limonene at 40° C. for 3 h 30 with stirring (plant matter/solvent ratio: 1/10). The particles are removed by filtration in order to obtain a solution laden with advantageous compounds, from which the d-limonene is subsequently removed in the absence of water by rectification under very high vacuum (1 mmHg, i.e. 133.2 Pa). The d-limonene is evaporated and then recycled, using a condenser maintained at 0° C., for subsequent extraction operations, while the nonsaponifiable compounds are recovered in the extractor.

198.9 g of extract rich in lipid compounds, in nonsaponifiable compounds and in caffeine are thus obtained. The extraction yield, expressed with respect to the dry weight of plant, is 13.26%. The extract thus obtained comprises 0.915% of nonsaponifiable compounds and 2.49% of caffeine.

The content of tannins, determined by the method described in the European Pharmacopeia 5.02 notice “Determination of tannins in herbal drugs (2.8.14.)”, is less than 0.1%.

### EXAMPLE No. 3

### Extraction of Curcuminoids from Turmeric According to the Process of the Invention in Comparison with an Extraction Method Using Hexane

Turmeric (Curcuma longa L.) rhizomes contain 8% of curcuminoids. 1 kg of finely ground turmeric powder of biological grade is macerated in 10 liters of d-limonene at 40° C. for 3 h 30 with stirring (plant matter/solvent ratio: 1/10).

The particles are removed by filtration in order to obtain a solution very strongly orange in color, from which the d-limonene is subsequently removed by azeotropic steam distillation, as described in example 1.

Approximately 12.5 liters of water are evaporated in order to remove the d-limonene.

81.5 g of extract rich in curcuminoids are thus obtained. The extraction yield, expressed with respect to the dry weight of plant, is 8.15%.

The same maceration and then desolvation operations can be carried out with different solvents or mixtures of solvents and the extraction yields as follows, expressed with respect to the dry weight of plant, are obtained:

| | |
|---|---|
| Solvent 1: hexane | 7.86% |
| Solvent 2 (restatement): d-limonene | 8.15% |
| Solvent 3: d-limonene/95° alcohol mixture as a | 8.49% |
| 70/30 (v/v) mixture | |
| Solvent 4: d-limonene of biological grade | 8.36% |
| Solvent 5: α-pinene | 8.10% |
| Solvent 6: α-pinene of biological grade | 8.55% |
| Solvent 7: d-limonene/α-pinene 70/30 (v/v) mixture: | 8.85% |

The extraction yields obtained with monoterpene hydrocarbons are better than that obtained with hexane.

The best extraction yields are obtained with biological α-pinene, the d-limonene/α-pinene 70/30 (v/v) mixture, biological d-limonene and the d-limonene/95° alcohol mixture as a 70/30 (v/v) mixture. A synergistic effect is observed with the d-limonene/α-pinene 70/30 (v/v) mixtures.

The extracts thus obtained comprise less than 0.05% of residual solvent, this limit being unacceptable for the extract obtained using hexane.

The content of tannins, determined by the method described in the European Pharmacopeia 5.02 notice “Determination of tannins in herbal drugs (2.8.14.)”, is less than 0.1%.

The above method thus makes it possible to selectively extract curcuminoids and to concentrate them.

During the maceration stage, it is not necessary to replace the solvent as the d-limonene virtually completely exhausts turmeric: a single extraction stage is necessary. It is also possible to extract curcuminoids from turmeric using a continuous countercurrentwise extraction device.

It is possible to proceed in an identical fashion in order to extract carotenoids from plants or lycopene from the tomato, for example.

Study of the antioxidant activity of the monoterpene extract:

The antioxidant activity of the turmeric extract obtained with solvent No. 2 (d-limonene) were studied using the xanthine/xanthine oxidase test according to the modified method of Rice Evans et al. (Techniques in free radical research, 1991, Elsevier, Paris) and Masaki et al. (Antioxygen scavenging activity of plant extracts, Biol. Pharm. Bull., 1995, 18 (1), 162-166).

The turmeric monoterpene extract exhibits an antioxidant activity (IC50=0.002%) which is three times greater than a hexane rosemary extract (Rosmarinus officinalis L; IC50=0.006%) and twenty times greater than allopurinol (IC50=0.06%), another reference antioxidant (n=10).

### EXAMPLE No. 4

### Extraction of Helenalin from Arnica According to the Process of the Invention, in Comparison with an Extraction Method Using Hexane

Arnica montana flower heads comprise 0.4% of sesquiterpene lactones (including 0.2-0.5% of helenalin in the plant) which are held responsible for the antiinflammatory and healing activity of Arnica. 200 g of whole Arnica (Arnica montana) flowering tops of biological grade are macerated in 5 liters of d-limonene at 40° C. for 3 h 30 with stirring (plant matter/solvent ratio: 1/25).

The particles are removed by filtration in order to obtain a solution strongly yellow in color, from which the d-limonene is subsequently removed by azeotropic steam distillation, as described in example 1.

Approximately 6.3 liters of water are evaporated in order to remove the d-limonene.

18.44 g of extract enriched in sesquiterpene lactones are thus obtained. The extraction yield, expressed with respect to the dry weight of plant, is 9.22%.

The same maceration and then desolvation operations can be carried out with different solvents or mixtures of solvents and the extraction yields as follows, expressed with respect to the dry weight of plant, are obtained:

| | |
|---|---|
| Solvent 1: hexane | 5.84% |
| Solvent 2 (restatement): d-limonene | 9.22% |
| Solvent 3: d-limonene/95° alcohol mixture as a | 9.48% |
| 70/30 (v/v) mixture | |
| Solvent 4: d-limonene of biological grade | 10.38% |
| Solvent 5: α-pinene | 7.81% |
| Solvent 6: α-pinene of biological grade | 9.11% |
| Solvent 7: d-limonene/α-pinene 70/30 (v/v) mixture: | 10.47% |

The extraction yields obtained with monoterpene hydrocarbons are better than that obtained with hexane.

The best extraction yields are obtained with biological α-pinene, the d-limonene/α-pinene 70/30 (v/v) mixture, biological d-limonene and the d-limonene/95° alcohol mixture as a 70/30 (v/v) mixture. A synergistic effect is observed with the d-limonene/α-pinene 70/30 (v/v) mixtures.

The extracts thus obtained comprise less than 0.05% of residual solvent, this limit being unacceptable for the extract obtained using hexane.

The content of tannins, determined by the method described in the European Pharmacopeia 5.02 notice “Determination of tannins in herbal drugs (2.8.14.)”, is less than 0.1%.

The above method thus makes it possible to selectively extract sesquiterpene lactones and to concentrate them.

During the maceration stage, it is not necessary to replace the solvent as the d-limonene virtually completely exhausts Arnica: a single extraction stage is necessary. It is also possible to extract sesquiterpene lactones from Arnica using a continuous countercurrentwise extraction device.

### EXAMPLE No. 5

### Simultaneous Extraction of Nonsaponifiable Compounds and Caffeine from Green Coffee in a Reduced Time by the process According to the Invention and Irradiation Using Microwaves

A cylindrical glass cell, around which microwave generators (2000-3000 MHz) are positioned, is filled with 10 g of coarsely ground green coffee and 50 g of d-limonene. The cell receives a microwave UHF power of 100 W for 2 minutes 30 seconds. The solution, which is maintained at a temperature of less than 40° C., rapidly turns green-brown in color and is then filtered. The d-limonene is removed as described in example 2.

1.34 g of extract rich in lipid compounds, in nonsaponifiable compounds and in caffeine are thus obtained in a very short time. The extraction yield, expressed with respect to the dry weight of plant, is 13.4%.

The extract thus obtained comprises 0.917% of nonsaponifiable compounds and 2.51% of caffeine.

The content of tannins, determined by the method described in the European Pharmacopeia 5.02 notice “Determination of tannins in herbal drugs (2.8.14.)”, is less than 0.1%.

The extraction kinetics can be greatly improved by irradiating the plant with microwaves. Because of their properties, d-limonene and α-pinene absorb relatively little microwave radiation. They thus remain at a reduced temperature while facilitating the solubilization of the active compounds.

### EXAMPLE No. 6

### Extraction of Curcuminoids from Turmeric in a Reduced Time by the Process According to the Invention and Irradiation Using Microwaves

10 g of turmeric powder and 50 g of d-limonene are employed according to a procedure identical to that described in example 5.

0.856 g of extract rich in curcuminoids is obtained in a very short time. The extraction yield, expressed with respect to the dry weight of plant, is 8.56%.

### EXAMPLE No. 7

### Extraction of Sesquiterpene Lactones from Arnica in a Reduced Time by the Process According to the Invention and Irradiation Using Microwaves

5 g of Arnica flowering tops and 50 g of d-limonene are employed according to a procedure identical to that described in example 5.

0.473 g of extract rich in sesquiterpene lactones is thus obtained. The extraction yield, expressed with respect to the dry weight of plant, is 9.47%.

### EXAMPLE No. 8

### Extraction of Alkaloids from the Celandine (Chelidonium majus) by the Process According to the Invention

50 g of dried and ground celandine (Chelidonium majus L.) roots and radicles are macerated in 500 ml of d-limonene at 40° C. for 3 h 30 with stirring (plant matter/solvent ratio: 1/10).

The particles are removed by filtration in order to obtain a solution laden with advantageous compounds, from which the d-limonene is subsequently removed in the absence of water by rectification under very high vacuum (133.2 Pa), as described in example 2.

1.05 g of syrupy extract are thus obtained, to which 100 ml of N/10 sulfuric acid are added to dissolve the alkaloids, followed by basification using 130 ml of N/10 aqueous ammonia. After filtration, the analysis of the aqueous alkaline solution according to the method of the European Pharmacopeia shows that an alkaloid solution comprising 0.75% of total alkaloids, expressed as chelidonine, is obtained.

Thin layer chromatographic analysis according to the method described by Lavenir and Paris (Sur les alcaloïdes de la Chélidoine (Chelidonium majus L.). Répartitiondans les divers organes, isolement de la stylopine á partir des fruits [On the alkaloids of celandine (Chelidonium majus L.). Distribution in the various organs, isolation of stylopine from the fruits]. Annales Pharmaceutiques Françaises, 1965, 23 (5), 307-312) makes it possible to reveal the presence of the various typical alkaloids of celandine in the extract thus obtained according to the following decreasing proportions: chelidonine, chelerythrine, sanguinarine, coptisine and berberine.

### EXAMPLE No. 9

### Extraction of Chelidonine by the Process According to the Invention

100 g of dried and ground celandine (Chelidonium majus L.) roots and radicles are macerated in 1 liter of d-limonene at 40° C. for 3 h 30 with stirring (plant matter/solvent ratio: 1/10).

The particles are removed by filtration in order to obtain a solution laden with advantageous compounds, from which the d-limonene is subsequently removed in the absence of water by rectification under very high vacuum (133.2 Pa), as described in example 2.

21.6 g of syrupy extract are thus obtained, to which 100 ml of N/10 sulfuric acid heated to 50° C. are added. After cooling, 16.2 g of chelidonine hydrochloride crystals are recovered by filtration.

### EXAMPLE No. 10

### Extraction of Sanguinarine from Bloodroot Rhizomes by a Process According to the Invention

100 g of dried and ground bloodroot (Sanguinaria canadensis L.) rhizomes are macerated in 1 liter of d-limonene at 40° C. for 3 h 30 with stirring (plant matter/solvent ratio: 1/10).

The particles are removed by filtration in order to obtain a solution laden with advantageous compounds, from which the d-limonene is subsequently removed in the absence of water by rectification under very high vacuum (133.2 Pa), as described in example 2.

24.5 g of syrupy extract are thus obtained, to which 100 ml of N/10 sulfuric acid heated to 50° C. are added. After cooling, 18.7 g of sanguinarine hydrochloride crystals are recovered by filtration. These crystals can be dissolved in alcohol or in numerous excipients.

### EXAMPLE No. 11

### Extraction of Free Xanthones from Gentian by a Process According to the Invention

100 g of dried and ground gentian (Gentiana lutea L.) rhizomes are macerated in 1 liter of d-limonene at 40° C. for 3 h 30 with stirring (plant matter/solvent ratio: 1/10).

The particles are removed by filtration in order to obtain a solution very strongly yellow in color, from which the d-limonene is subsequently removed by azeotropic steam distillation, as described in example 1.

Approximately 1.45 liters of water are evaporated in order to remove the d-limonene.

4.51 g of extract rich in xanthones are thus obtained. The extraction yield, expressed with respect to the dry weight of plant, is 4.51%.

The content of tannins, determined by the method described in the European Pharmacopeia 5.02 notice “Determination of tannins in herbal drugs (2.8.14.)”, is less than 0.1%.

The HPLC analysis according to Carnat et al. (Journal of the Science of Food and Agriculture, 2005, 85 (4), 598-602, Influence of drying mode on iridoid bitter constituent levels in gentian root) makes it possible to show the presence of 3% of xanthones (gentisin, isogentisin) and 0.5% of glycosides (gentioside) in the extract.

This exemplary embodiment again demonstrates a feature of the invention, that is to say that monoterpene hydrocarbons make possible the concomitant extraction of compounds having a lipophilic nature (example: xanthones) and compounds having a polar nature (example: glycosides), and their concentration, while excluding other components (for example, the iridoids responsible for the bitterness of gentian extracts) nevertheless present in a large amount in the starting material.

This method effectively makes possible the selective extraction of lipophilic compounds and compounds exhibiting an intermediate polarity.

The xanthone compounds and the glycosylated forms thus extracted from gentian can easily be incorporated in forms for oral administration to man which are devoid of bitterness.

### EXAMPLE No. 12

### Extraction of Parthenolides from Feverfew (Tanacetum Parthenium L.)

Tanacetum parthenium flower heads comprise 1.5% of sesquiterpene lactones (mainly parthenolide) which are held responsible for the antimigraine activity of feverfew.

100 g of feverfew flowering tops are macerated in 2.5 liters of d-limonene at 40° C. for 3 h 30 with stirring (plant matter/solvent ratio: 1/25).

The particles are removed by filtration in order to obtain a solution very strongly yellow in color, from which the d-limonene is subsequently removed by azeotropic steam distillation, as described in example 1.

Approximately 3.8 liters of water are evaporated in order to remove the d-limonene.

1.37 g (yield=1.37%) of extract rich in sesquiterpene lactones and comprising 0.83% of parthenolides by HPLC analysis according to the method of Zhou et al. (Rapid extraction and high performance liquid chromatographic determination of parthenolide in feverfew (Tanacetum parthenium), Journal of Agricultural and Food Chemistry, 47 (3), 10018-1022, 1999) are thus obtained.

The thin layer chromatographic analysis according to the method described by Wagner et al. (Plant Drug Analysis-A Thin Layer Chromatography, 1984, Springer Verlag, 1984, pp. 182-184) also makes it possible to demonstrate the presence of chlorogenic acid, flavanoid aglycones and apigenin 7-O-glucoside. The presence is also observed here of compounds having a lipophilic nature (sesquiterpene lactones) and compounds having a more polar nature (chlorogenic acid, flavonoid aglycones and apigenin glucoside).

The above examples can be applied to numerous other plants, for example liquorice, ginger and Serenoa repens, without this list being limiting.

The extracts according to the invention can be easily incorporated in liquid or semiliquid compositions, in particular liquid or semiliquid pharmaceutical or cosmetic compositions, in order to be provided in the pharmaceutical forms commonly used in human medicine and prepared according to the usual methods. The extract or extracts according to the invention can be incorporated in excipients commonly employed in pharmaceutical compositions, such as aqueous or nonaqueous vehicles, various wetting agents, preservatives or thickeners.

Another subject matter of the present invention is pharmaceutical and/or cosmetic compositions comprising, as active ingredient, an extract according to the invention and/or the process for the preparation of said compositions, characterized in that the extract or extracts according to the invention is/are mixed, according to methods known per se, with acceptable excipients or solvents, in particular pharmaceutically or cosmetically acceptable excipients or solvents.

As the extraction employs solvents of plant origin, the constituent or constituents of which are characterized and chemically defined, such as, for example, monoterpene hydrocarbons of biological grade, said extraction is a novel alternative technical solution for molecules which cannot be steam distilled and also to extraction by supercritical CO₂, and it is effective for a fairly wide range of molecules used in the cosmetics and pharmaceutical industries. The extracts obtained according to this process comprise compounds which exhibit a lipophilic nature or an intermediate polarity.

1. A single-phase extraction process, comprising:
bringing a plant material into contact with a solvent and/or a mixture of solvents of plant origin, the constituent or constituents of which are characterized and chemically defined,
removing the plant material, and
partially or completely removing the solvent and/or mixture of solvents, said solvent and/or at least one of the constituents of said mixture of solvents being chosen from the group consisting of terpene solvents, having a purity at least equal to 98%, and being poor in peroxides.

2. The process as claimed in claim 1, wherein the solvent and/or mixture of solvents is an isomer or a mixture of isomers of limonene and/or a pinene, alone or as a mixture.

3. The process as claimed in claim 2, wherein the solvent and/or mixture of solvents is a mixture of d-limonene and of α-pinene.

4. The process as claimed in claim 1, wherein the solvent is a mixture of d-limonene and of α-pinene, with at least 50% of d-limonene.

5. The process as claimed in claim 1, wherein the mixture comprises 70% of d-limonene and 30% of α-pinene.

6. The process as claimed in claim 1, wherein ethanol can be used as a mixture with at least one terpene solvent.

7. The process as claimed in claim 6, wherein the mixture is chosen from the groups formed by the following binary or ternary mixtures:
d-limonene/95′ ethanol: 70%/30%,
α-pinene/95′ ethanol: 70%/30%,
d-limonene/α-pinene/95′ ethanol: 50%/20%/30%.

8. The process as claimed in claim 1, wherein the solvent and/or at least one of the constituents of said mixture of solvents has been subjected to a preliminary purification stage.

9. The process as claimed in claim 8, wherein the purification stage is chosen from azeotropic steam distillation or from vacuum evaporation.

10. The process as claimed in claim 1, wherein the solvent and/or at least one of the constituents of said mixture of solvents exhibits a purity of greater than or equal to 98%.

11. The process as claimed in claim 10, wherein the solvent and/or at least one of the constituents of said mixture of solvents exhibits a purity of 99.95%.

12. The process as claimed in claim 1, wherein the solvent and/or at least one of the constituents of said mixture of solvents exhibits a level of peroxides of less than 15 meq/kg.

13. The process as claimed in claim 1, wherein the extraction temperature is between 40° C. and 45° C.

14. The process as claimed in claim 1, wherein a stage of irradiation with microwaves is carried out during the stage of bringing the plant material into contact with the solvent and/or mixture of solvents.

15. The process as claimed in claim 1, wherein the stage of removal of the solvent and/or mixture of solvents is a stage of azeotropic steam distillation.

16. The process as claimed in claim 1, wherein the stage of removal of the solvent and/or mixture of solvents is a vacuum molecular distillation.

17. The process as claimed in claim 1, wherein it is carried out with plant matter/solvent and/or mixture of solvents proportions by weight of 1/4 to 1/10.

18. A plant extract obtained by the extraction process as claimed in claim 1.

19. The plant extract as claimed in claim 18, wherein it is devoid of polyphenol and of anthocyan, in that it comprises at most 0.1% of tannins and in that it comprises a fraction of compounds having a lipophilic nature and/or a fraction of compounds having a polar nature.

20. The plant extract as claimed in claim 19, wherein the compounds having a lipophilic nature are chosen in particular from nonsaponifiable compounds.

21. The plant extract as claimed in claim 19, wherein the compounds having a polar nature are chosen from alkaloids, neoxanthins, glycolyzed xanthones, flavonoids or glycosylated flavonoids.

22. The plant extract as claimed in claim 18, the plant being green coffee, wherein it comprises at least 0.8% of nonsaponifiable compounds, at least 2.0% of caffeine and at most 0.1% of tannins.

23. The plant extract as claimed in claim 18, the plant being turmeric, wherein it comprises at least 8% of curcuminoids and at most 0.1% of tannins.

24. The plant extract as claimed in claim 18, the plant being Arnica montana flowers, wherein it comprises at least 5% of sesquiterpene lactones, expressed as helenalin, and at most 0.1% of tannins.

25. The plant extract as claimed in claim 18, the plant being celandine, wherein it comprises at least 0.75% of total alkaloids, expressed as chelidonine or crystals of chelidonine hydrochloride, and at most 0.1% of tannins.

26. The plant extract as claimed in claim 18, the plant being bloodroot, wherein it comprises crystals of sanguinarine hydrochloride and at most 0.1% of tannins.

27. The plant extract as claimed in claim 18, the plant being gentian, wherein it comprises at least 3% of free xanthones with at least 0.5% of gentioside and at most 0.1% of tannins.

28. The plant extract as claimed in claim 18, the plant being feverfew, wherein it comprises at least 0.83% of parthenolides, flavonoid aglycones, apigenin glucosides and at most 0.1% of tannins.
